(19) United States
(12) Patent Application Publication
(10) Pub. No.: US 2014/0294737 A1
(43) Pub. Date: Oct. 2, 2014

(54) TREATMENT OF RHINITIS

(71) Applicant: AMPIO PHARMACEUTICALS, INC., Greenwood Village, CO (US)

(72) Inventor: David Bar-Or, Englewood, CO (US)

(21) Appl. No.: 14/349,412

(22) PCT Filed: Oct. 5, 2012
(86) PCT No.: PCT/US2012/059061

§ 371 Date: Apr. 3, 2014

Related U.S. Application Data
(60) Provisional application No. 61/544,499, filed on Oct. 7, 2011.
(60) Provisional application No. 61/552,517, filed on Oct. 28, 2011.

(51) Int. Cl. A61K 31/58; A61M 11/02; A61M 15/00; A61K 45/06

(52) U.S. Cl. CPC A61K 31/58; A61K 45/06; A61M 11/02; A61M 15/009
USPC 424/45; 514/176; 514/171; 128/200.21

(57) ABSTRACT

The invention provides a method of treating rhinitis. The method comprises administering an effective amount of a danazol compound or a pharmaceutically acceptable salt thereof. The invention also provides a pharmaceutical product formulated for nasal administration. The product comprises a danazol compound or a pharmaceutically acceptable salt thereof.

### CROSS-REFERENCE TO RELATED APPLICATIONS

This application claims the benefit of priority under 35 U.S.C. §119(e) to U.S. Provisional Patent Application No. 61/544,499, filed Oct. 7, 2011 and U.S. Provisional Patent Application No. 61/552,517, filed Oct. 28, 2011. The entire disclosures of each U.S. Provisional Patent Applications Nos. 61/544,499 and 61/552,517 are incorporated herein by reference.

### FIELD OF INVENTION

The invention relates to a method of treating rhinitis. The method comprises administering an effective amount of a danazol compound or a pharmaceutically acceptable salt thereof. The invention also provides a pharmaceutical product comprising a danazol compound or a pharmaceutically-acceptable salt thereof.

### BACKGROUND

Rhinitis is caused by chronic or acute inflammation of the mucous membranes of the nose due to viruses, bacteria or irritants. The inflammation results in the generation of excessive amounts of mucous, commonly producing a runny nose, nasal congestion and post-nasal drip. Rhinitis is reported to affect more than 50 million people in the United States alone.

There are several types of rhinitis, including infectious rhinitis, allergic rhinitis and nonallergic rhinitis. Infectious rhinitis is caused by a viral or bacterial infection. Types of infectious rhinitis include the common cold and sinusitis.

Allergic rhinitis affects more than 20% of people worldwide and the prevalence increases annually. Allergic rhinitis causes impaired social life, sleep, school, and work. The quality of life of patients can be altered by the severity and duration of rhinitis. Allergic rhinitis is a proinflammatory immune response to outdoor or indoor allergens, such as dust or pollen. Symptoms can occur year-round or primarily be at certain times of the year, usually in the spring, summer or fall. The Allergic Rhinitis and its Impact on Asthma (ARIA) guidelines outlines the management of allergic rhinitis as allergen avoidance, patient education, pharmacotherapy, and allergen-specific immunotherapy. For pharmacotherapy, ARIA currently recommends intranasal, second-generation H1-antihistamines and an intranasal corticosteroid for moderate to severe persistent disease. See Bousquet et al., J. Allergy Clin. Immunol., 108(Suppl 5):S147-334 (2001) and Bousquet et al., Allergy, 63(Suppl. 86):8-160 (2008).

Nonallergic rhinitis is rhinitis that is not triggered by allergens or infectious agents. There is still much to be learned about nonallergic rhinitis, but it is thought that the triggers of it cause dilation of the blood vessels in the lining of the nose, which results in swelling and drainage. Types of nonallergic rhinitis include vasomotor, autonomic, hormonal, drug-induced, atrophic and gustatory rhinitis and rhinitis medicamentosa. Triggers of vasomotor rhinitis include smells, fumes, smoke, dust and temperature changes, and vasomotor rhinitis can coexist with allergic rhinitis. Rhinitis medicamentosa is a condition of rebound nasal congestion brought on by extended use of topical decongestants.

### SUMMARY OF THE INVENTION

One embodiment of the invention relates to a method of treating rhinitis by administering an effective amount of a danazol compound or a pharmaceutically acceptable salt thereof, to an animal in need thereof. In one aspect, the rhinitis is allergic rhinitis.

In one aspect, the effective amount of the danazol compound or the pharmaceutically acceptable salt thereof is from about 100 μg to about 3000 μg per day. In another aspect, the effective amount of the danazol compound or the pharmaceutically acceptable salt thereof is from about 500 μg to about 1500 μg per day.

In yet other aspects, the administration of the danazol compound or a pharmaceutically acceptable salt thereof, is commenced within 24 hours of diagnosis of rhinitis. In still other aspects, the administration of the danazol compound or a pharmaceutically acceptable salt thereof, is commenced at the appearance of one or more early signs of, or a predisposition to develop, rhinitis. One or more early signs of rhinitis can be rhinorrhea, nasal congestion, nasal itching and sneezing.

In still another aspect, the danazol compound is danazol.

In yet another aspect, the danazol compound or a pharmaceutically acceptable salt thereof is administered in combination with a second drug suitable for treating rhinitis. For example, the second drug can be selected from antihistamines, decongestants, anti-inflammatories, mast cell stabilizers, leukotriene modifiers and IgE blockers.

Another embodiment of the invention relates to a pharmaceutical product, which can comprise about 0.1% (w/v) of a danazol compound or a pharmaceutically-acceptable salt thereof formulated for administration by a route selected from inhalation, insufflation and nasal administration to the nose. In one aspect, the danazol compound or a pharmaceutically-acceptable salt thereof formulated for administration by inhalation is packaged in a device selected from insufflators, nebulizers, pressurized packs, squeeze bottle, a syringe, a dropper, a spray device, an atomizer device, and an aerosolizer. In one aspect, the pressurized pack can comprise a propellant selected from dichlorodifluoromethane, trichlorofluoromethane, dichlorotetrafluoroethane, and carbon dioxide.

In still another aspect, the danazol compound or a pharmaceutically-acceptable salt thereof formulated for administration by inhalation or insufflation comprises a powder mix of the danazol compound or pharmaceutically-acceptable salt thereof and a powder base. In one aspect, the powder mix can be in a dosage form selected from capsules, cartridges, gelatin packs and blister packs. The powder mix can be delivered by a device selected from an inhalator, insufflator and metered-dose inhaler

In yet another aspect, the danazol compound or a pharmaceutically-acceptable salt thereof formulated for nasal administration is in a form of drops or sprays. The drops or sprays can be contained within an intranasal delivery system. In one aspect, the intranasal delivery system comprises an atomizing device. In one aspect, the atomizing device comprises a bottle and pump. In a preferred aspect, the pump is a metered dose pump. The metered dose pump can deliver an intranasal volume of the danazol compound or a pharmaceutically-acceptable salt thereof. In one aspect, the metered dose pump can deliver an intranasal volume of about 0.15 ml of the danazol compound or pharmaceutically-acceptable salt thereof per pump. In still another pharmaceutical product further comprises an aqueous or non-aqueous base comprising one or more agents selected from dispersing agents, solubilizing agents, and suspending agents.

In still another aspect, the danazol compound or pharmaceutically-acceptable salt thereof is formulated for nasal administration in a form selected from ointments, gels and creams. In one aspect, pharmaceutical product further comprises excipients selected from animal fats, vegetable fats, oils, waxes, paraffins, starch, tragacanth, cellulose derivatives, polyethylene glycol, silicones, bentonites, silicic acid, talc, zinc oxide and mixtures thereof. In still another aspect, the pharmaceutical product further comprises an absorption or permeation enhancer. In yet another aspect, the pharmaceutical product further comprises a thickening agent or viscosity enhancer to increase the residence time of the danazol compound or pharmaceutically-acceptable salt thereof in the nose. In still another aspect, the pharmaceutical product further comprises a pharmaceutically-acceptable carrier. In one aspect, the danazol compound is danazol.

### BRIEF DESCRIPTION OF THE DRAWINGS

FIG. 1 shows the mean severity change in the reflective total nasal symptom score (rTNSS) obtained by analysis using the Student's t-test comparing treatment with either a placebo or danazol. “AM rTNSS” represents scores assessed by the subjects in the morning; PM rTNSS” represents scores assessed by the subjects in the evening. Number of subjects screened, n=21; number of subjects randomized, n=20; number of subjects treated with placebo, n=10 and number of subjects treated with danazol, n=10.

FIG. 2 shows the mean change in reflective symptoms using the TNSS analysis described in FIG. 1. The subjects assessed the following symptoms: runny nose, stuffy nose, itchy nose and sneezing.

FIG. 3 shows the mean severity change in the instantaneous total nasal symptom score (iTNSS) obtained by analysis using the Student's t-test comparing treatment with either a placebo or danazol. “AM iTNSS” represents scores assessed by the subjects in the morning; “PM iTNSS” represents scores assessed by the subjects in the evening. Number of subjects screened, n=21; number of subjects randomized, n=20; number of subjects treated with placebo, n=10 and number of subjects treated with danazol, n=10.

FIG. 4 shows the mean change in instantaneous symptoms using the TNSS analysis described in FIG. 3. The subjects assessed the following symptoms: runny nose, stuffy nose, itchy nose and sneezing.

FIG. 5 shows the median (IQR) severity change in the reflective total nasal symptom score (rTNSS) obtained by analysis using the Wilcoxon rank-sum test comparing treatment with either a placebo or danazol. “AM rTNSS” represents scores taken by the subjects in the morning; “PM rTNSS” represents scores taken by the subjects in the evening. Number of subjects screened, n=21; number of subjects randomized, n=20; number of subjects treated with placebo, n=10 and number of subjects treated with danazol, n=10.

FIG. 6 shows the median change in reflective symptoms using the TNSS analysis described in FIG. 5. The subjects assessed the following symptoms: runny nose, stuffy nose, itchy nose and sneezing.

FIG. 7 shows the median (IQR) severity change in the instantaneous total nasal symptom score (iTNSS) obtained by analysis using the Wilcoxon rank-sum test comparing treatment with either a placebo or danazol. “AM iTNSS” represents scores taken by the subjects in the morning; “PM iTNSS” represents scores taken by the subjects in the evening. Number of subjects screened, n=21; number of subjects randomized, n=20; number of subjects treated with placebo, n=10 and number of subjects treated with danazol, n=10.

FIG. 8 shows the median change in instantaneous symptoms using the TNSS analysis described in FIG. 7. The subjects assessed the following symptoms: runny nose, stuffy nose, itchy nose and sneezing.

### DETAILED DESCRIPTION OF THE INVENTION

The invention provides for a method of treating rhinitis, including but not limited to infectious rhinitis, allergic rhinitis and nonallergic rhinitis. The method comprises administering to an animal in need thereof an effective amount of a danazol compound, prodrug or pharmaceutically-acceptable salt thereof.

Allergic rhinitis is a proinflammatory immune response to outdoor or indoor allergens, such as dust or pollen. Nonallergic rhinitis is rhinitis that is not triggered by allergens or infectious agents. Types of nonallergic rhinitis include but are not limited to vasomotor, autonomic, hormonal, drug-induced, atrophic and gustatory rhinitis and rhinitis medicamentosa.

As used herein, “a danazol compound” means danazol, prodrugs of danazol and pharmaceutically acceptable salts of danazol and its prodrugs.

Danazol (17α-pregna-2,4-dien-20-yno[2,3-d]-isoxazol-17β-ol) is a known synthetic steroid hormone. Its structure is:

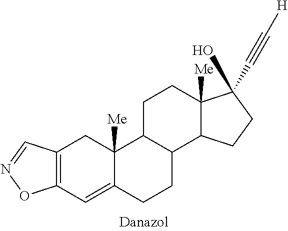 [SMILES: C#C[C@]1(O)CCC2C3CCC4=Cc5oncc5C[C@]4(C)C3CC[C@@]21C]

Methods of making danazol are known in the art. See e.g., U.S. Pat. No. 3,135,743, and GB Patent No. 905,844. Also, danazol is available commercially from many sources, including Barr Pharmaceuticals, Inc., Lannett Co., Inc., sanofi-aventis Canada, Sigma-Aldrich, and Parchem Trading Ltd.

“Prodrug” means any compound which releases an active parent drug (danazol in this case) in vivo when such prodrug is administered to an animal. Prodrugs of danazol include danazol wherein the hydroxyl group is bonded to any group that may be cleaved in vivo to generate the free hydroxyl. Examples of danazol prodrugs include esters (e.g., acetate, formate, and benzoate derivatives) of danazol.

The pharmaceutically-acceptable salts of danazol and its prodrugs include conventional non-toxic salts, such as salts derived from inorganic acids (such as hydrochloric, hydrobromic, sulfuric, phosphoric, nitric, and the like), organic acids (such as acetic, propionic, succinic, glycolic, stearic, lactic, malic, tartaric, citric, glutamic, aspartic, benzoic, salicylic, oxalic, ascorbic acid, and the like) or bases (such as the hydroxide, carbonate or bicarbonate of a pharmaceutically-acceptable metal cation or organic cations derived from N,N-dibenzylethylenediamine, D-glucosamine, or ethylenediamine). The salts are prepared in a conventional manner, e.g., by neutralizing the free base form of the compound with an acid. In particular, isoxazoles, such as danazol, are weakly basic substances and will form acid-addition salts upon addition of strong acids and quaternary ammonium salts upon addition of esters of strong acids (e.g., an ester of a strong inorganic or organic sulfonic acid, preferably a lower-alkyl, lower alkenyl or lower aralkyl ester, such as methyl iodide, ethyl iodide, ethyl bromide, propyl bromide, butyl bromide, allyl bromide, methyl sulfate, methyl benezenesulfonate, methyl-p-toluene-sulfonate, benzyl chloride and the like). See U.S. Pat. No. 3,135,743.

As noted above, a danazol compound can be used to treat rhinitis. To do so, the danazol compound is administered to an animal in need of treatment. Preferably, the animal is a mammal, such as a rabbit, goat, dog, cat, horse or human. Most preferably, the animal is a human.

Effective dosage forms, modes of administration and dosage amounts for the compounds of the invention (i.e., danazol, a prodrug of danazol or a pharmaceutically-acceptable salt of either one of them) may be determined empirically using the guidance provided herein. It is understood by those skilled in the art that the dosage amount will vary with the particular disease or condition to be treated, the severity of the disease or condition, the route(s) of administration, the duration of the treatment, the identity of any other drugs being administered to the animal, the age, size and species of the animal, and like factors known in the medical and veterinary arts. In general, a suitable daily dose of a compound of the present invention will be that amount of the compound which is the lowest dose effective to produce a therapeutic effect. However, the daily dosage will be determined by an attending physician or veterinarian within the scope of sound medical judgment. If desired, the effective daily dose may be administered as two, three, four, five, six or more sub-doses, administered separately at appropriate intervals throughout the day. Administration of the compound should be continued until an acceptable response is achieved. Such an acceptable response may be for example when the symptoms of rhinitis are reduced and/or when the symptoms of rhinitis are no longer detected by the subject.

Danazol compounds can be used in the practice of the present invention at optimum doses that are about 100-1500 times lower than those amounts currently administered to patients for the treatment of other diseases and conditions (typically 200-800 mg/day for an adult human). Uses of these lower doses of danazol compounds should avoid any significant side effects, perhaps all side effects, which will be especially advantageous for early or prophylatic treatment of diseases and conditions according to the present invention. In particular, an effective dosage amount of a danazol compound administered nasally for treating rhinitis will be from about 100 μg/day to about 3000 μg/day (half in each nostril), preferably about 200 μg/day to about 2000 μg/day, most preferably about 500 μg/day to about 1500 μg/day. Effective dosage amounts can be a range with a lower end point of about 10 μg/day, about 20 μg/day, about 30 μg/day, about 40 μg/day, about 50 μg/day, about 60 μg/day, about 70 μg/day, about 80 μg/day, about 90 μg/day, about 100 μg/day, about 200 μg/day, about 300 μg/day, about 400 μg/day, about 500 μg/day, about 600 μg/day, about 700 μg/day, about 800 μg/day, about 900 μg/day, about 1000 μg/day. Effective dosage amounts can be a range with an upper endpoint of about 5000 μg/day, about 4000 μg/day, about 3000 μg/day, about 2800 μg/day, about 2600 μg/day, about 2400 μg/day, about 2200 μg/day, about 2000 μg/day, about 1900 μg/day, about 1800 μg/day, about 1700 μg/day, about 1600 μg/day, about 1500 μg/day, about 1400 μg/day, about 1300 μg/day, about 1200 μg/day, about 1100 μg/day, about 1000 μg/day. Additionally, when the danazol compounds are administered as a spray composition and/or as drop composition, the effective dosage amounts can be a range with a lower end point of about 0.01% (weight/volume (w/v)), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.10% (w/v), about 0.11% (w/v), about 0.12% (w/v), about 0.13% (w/v), about 0.14% (w/v), about 0.15% (w/v), about 0.16% (w/v), about 0.17% (w/v), about 0.18% (w/v), about 0.19% (w/v), about 0.20% (w/v), about 0.25% (w/v), about 0.30% (w/v), about 0.35% (w/v), about 0.40% (w/v), about 0.45% (w/v), about 0.50% (w/v). Most preferably about 0.1% (w/v). Also, when the danazol compounds are administered as a spray composition and/or as drop composition, the effective dosage amounts can be a range with a upper end point about 1.0% (w/v), about 0.95% (w/v), about 0.90% (w/v), about 0.85% (w/v), about 0.80% (w/v), about 0.75% (w/v), about 0.70% (w/v), about 0.65% (w/v), about 0.60% (w/v), about 0.55% (w/v).

When administered systemically, an effective dosage amount will also be that amount that will result in a concentration in a relevant fluid (e.g., blood) from about 0.0001 μM to about 5 μM, from about 0.001 μM to about 4 μM, from about 0.01 μM to about 3 μM, preferably from about 0.1 μM to about 1.0 μM, more preferably from about 0.1 μM to about 0.5 μM, most preferably about 0.1 μM. An effective dosage amount will also be that amount that will result in a concentration in the tissue or organ to be treated of about 0.17% (weight/weight) or less, preferably from 0.00034% to 0.17%, most preferably 0.0034% to 0.017%. When given orally to an adult human, the dose will preferably be from about 1 ng/day to about 100 mg/day, more preferably the dose will be from about 1 mg/day to about 100 mg/day, most preferably the dose will be from about 10 mg/day to about 90 mg/day, preferably given in two equal doses per day. Further, danazol is expected to accumulate in cells and tissues, so that an initial (loading) dose (e.g. 100 mg per day) may be reduced after a period of time (e.g., 2-4 weeks) to a lower maintenance dose (e.g. 1 mg per day) which can be given indefinitely without significant side effects, perhaps without any side effects.

The administration of the danazol compound may be commenced within 24 hours of diagnosis of rhinitis. The administration of the danazol compound may be commenced at the appearance of one or more early signs of, or a predisposition to develop, rhinitis. The early signs of rhinitis include but are not limited torhinorrhea, nasal congestion, nasal itching and sneezing.

The compounds of the present invention (i.e., danazol, prodrugs thereof and pharmaceutically-acceptable salts of either of them) may be administered to an animal patient for therapy by any suitable route of administration, including orally, nasally, parenterally (e.g., intravenously, intraperitoneally, subcutaneously or intramuscularly), transdermally, intraocularly and topically (including buccally and sublingually). Preferred is oral, ocular or nasal administration for any disease or condition treatable according to the invention. Especially preferred is nasal administration.

While it is possible for a compound of the present invention to be administered alone, it is preferable to administer the compound as a pharmaceutical formulation (composition). The pharmaceutical compositions of the invention comprise a compound or compounds of the invention as an active ingredient in admixture with one or more pharmaceutically-acceptable carriers and, optionally, with one or more other compounds, drugs or other materials. Each carrier must be “acceptable” in the sense of being compatible with the other ingredients of the formulation and not injurious to the animal. Pharmaceutically-acceptable carriers are well known in the art. Regardless of the route of administration selected, the compounds of the present invention are formulated into pharmaceutically-acceptable dosage forms by conventional methods known to those of skill in the art. See, e.g., Remington's Pharmaceutical Sciences.

Formulations of the invention suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a compound or compounds of the present invention as an active ingredient. A compound or compounds of the present invention may also be administered as bolus, electuary or paste.

In solid dosage forms of the invention for oral administration (capsules, tablets, pills, dragees, powders, granules and the like), the active ingredient (i.e., danazol, a prodrug of danazol, a pharmaceutically-acceptable salt of either one of them, or combinations of the foregoing) is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient only, or preferentially, in a certain portion of the gastrointestinal tract, optionally, in a delayed manner. Examples of embedding compositions which can be used include polymeric substances and waxes. The active ingredient can also be in microencapsulated form.

Liquid dosage forms for oral administration of the compounds of the invention include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active ingredient, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical formulations and products include those suitable for administration by inhalation or insufflation or for nasal administration. For administration to the upper (nasal) or lower respiratory tract by inhalation, the compounds of the invention are conveniently delivered from a device for inhalation delivery such as an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of one or more compounds of the invention and a suitable powder base, such as lactose or starch. The powder composition may be presented in unit dosage form in, for example, capsules or cartridges, or, e.g., gelatin or blister packs from which the powder may be administered with the aid of an inhalator, insufflator or a metered-dose inhaler.

For nasal administration, compounds of the invention may be administered by means of nose drops or a liquid spray, such as by means of a plastic bottle spray or atomizer or metered-dose inhaler. Liquid sprays are conveniently delivered from pressurized packs.

Nose drops may be formulated with an aqueous or nonaqueous base also comprising one or more dispersing agents, solubilizing agents or suspending agents. Drops can be delivered by means of a simple eye dropper-capped bottle or by means of a plastic bottle adapted to deliver liquid contents dropwise by means of a specially shaped closure.

Ointments, gels and creams can also be used for nasal administration of the compounds of the invention. The active ingredient may be mixed under sterile conditions with a pharmaceutically-acceptable carrier, and with any buffers, or propellants which may be required. The ointments, creams and gels may contain, in addition to the active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Dosage forms for topical administration or for transdermal administration of compounds of the invention include powders, sprays, ointments, pastes, creams, lotions, gels, solutions, patches, drops and inhalants. The active ingredient may be mixed under sterile conditions with a pharmaceutically-acceptable carrier, and with any buffers, or propellants which may be required. The ointments, pastes, creams and gels may contain, in addition to the active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the active ingredient, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane. Transdermal patches have the added advantage of providing controlled delivery of compounds of the invention to the body. Such dosage forms can be made by dissolving, dispersing or otherwise incorporating one or more compounds of the invention in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel. A drug-impregnated solid carrier (e.g., a dressing) can also be used for topical administration.

Pharmaceutical compositions of this invention suitable for parenteral administrations comprise one or more compounds of the invention in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents. Also, drug-coated stents may be used.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monosterate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug is accomplished by dissolving or suspending the drug in an oil vehicle.

Injectable depot forms are made by forming microencapsule matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

A danazol compound may be given alone to treat rhinitis. Alternatively, the danazol compound may be given in combination with one or more other treatments or drugs suitable for treating the rhinitis. For instance, the danazol compound can be administered prior to, in conjunction with (including simultaneously with), or after the other treatment or drug. In the case of another drug, the drug and the danazol compound may be administered in separate pharmaceutical compositions or as part of the same pharmaceutical composition. Suitable other drugs include antihistamines, decongestants, anti-inflammatories (steroidal and nonsteroidal), mast cell stabilizers, leukotriene modifiers and IgE blockers. Specific suitable drugs include fexofenadine, doxylamine, diphenhydramine, triprolidine, loratidine, cetirizine, pseudophedrine, phenylephrine, aspirin, ibuprofen, naproxen, prednisone, prednisolone, and methylprednisolone.

Suitable drugs for inclusion in a nasal spray are steroids (such as fluticasone propionate, mometasone, budesonite, flunisolide, triamcinolone and beclomethasone), antihistamines (such as azelastine), anticholinergics (such as ipratproium) and mast cell stabilizers (such as cromolyn).

As used herein, “a” or “an” means one or more.

As used herein, “comprises” and “comprising” include within their scope all narrower terms, such as “consisting essentially of” and “consisting of” as alternative embodiments of the present invention characterized herein by “comprises” or “comprising”. In regard to use of “consisting essentially of”, this phrase limits the scope of a claim to the specified steps and materials and those that do not materially affect the basic and novel characteristics of the invention disclosed herein.

Additional objects, advantages and novel features of the present invention will become apparent to those skilled in the art by consideration of the following non-limiting examples. The following experimental results are provided for purposes of illustration and are not intended to limit the scope of the invention.

### EXAMPLE

A Phase Ib randomized, double-blinded, placebo-controlled, parallel group study was performed to evaluate the efficacy of danazol for treating allergic rhinitis in adult humans. Briefly, the study was performed as follows.

The subjects were male or female humans, 18-65 years of age, with a history of allergic rhinitis that had been receiving therapy (continuous or intermittent) for more than 1 year. Each subject had a demonstrated sensitivity to at least one seasonal allergen (tree/grass pollen) known to induce allergic rhinitis through a standard skin test. Each subject had a minimum subject-reported reflective Total Nasal Symptom Score (rTNSS) of ≧5 on the day of the Screening Visit (day 1).

During a seven-day wash out period (days 1-7), the subjects received no treatment of any type and recorded their symptoms twice a day (in the morning before bathing, consumption of food or beverages or strenuous activities and 12 hours later) using the following scale:
0=absent (no sign/symptom present)
1=mild (sign/symptom clearly present, but minimal awareness; easily tolerated)
2=moderate (definite awareness of sign/symptom that is bothersome but tolerable)
3=severe (sign/symptom that is hard to tolerate; causes interference with activities of daily living and/or sleeping)

The subjects were randomized into treatment groups (danazol or placebo) after the wash out period (on day 8).

Treatment was begun on day 8 and continued through day 21 (14 days total). During treatment, the subjects again recorded their symptoms twice daily (as described above and before administration of the test medications in the morning) using the above scale. The subjects administered 0.3 ml of one of the test medications, 0.15 ml per nostril, once daily in the morning, immediately after recording their symptoms. The first administration of test medications was supervised by test site personnel. The test medications were 0.1% (w/v) danazol intranasal spray and placebo spray (nonmedicinal components in identical intranasal spray format). Efficacy of the test medications was assessed as follows:

The measures of effectiveness in this study included the subject-reported Total Nasal Symptom Score (TNSS). The TNSS is defined as the sum of the subject-reported symptom scores for the four nasal symptoms: rhinorrhea (runny nose), nasal congestion, nasal itching, and sneezing. Each score is assessed on a severity scale ranging from 0 to 3 as defined above.

The subjects were asked to assess both reflective TNSS (i.e., an evaluation of symptom severity over the past 12 hours prior to the recording of the score) and instantaneous TNSS (i.e., an evaluation of the symptom severity over the last 10 minutes).

The reflective and instantaneous TNSS are defined as the sum of the subject-reported symptom scores for the four nasal symptoms. Each subject recorded the symptom scores in the subject's diary. For each score, information recorded in the diary included the following:
rhinorrhea (runny nose)
nasal congestion
nasal itching
sneezing The severity scale for each symptom evaluation is given above.

Mean reflective (r) and instantaneous (i) subject-reported total nasal symptom scores (TNSS) were calculated for each subject. The mean TNSS is the average of all AM and PM daily scores (each score is ranked on a scale from 0-12) during the baseline (baseline efficacy value) and the treatment period (double-blind efficacy value).

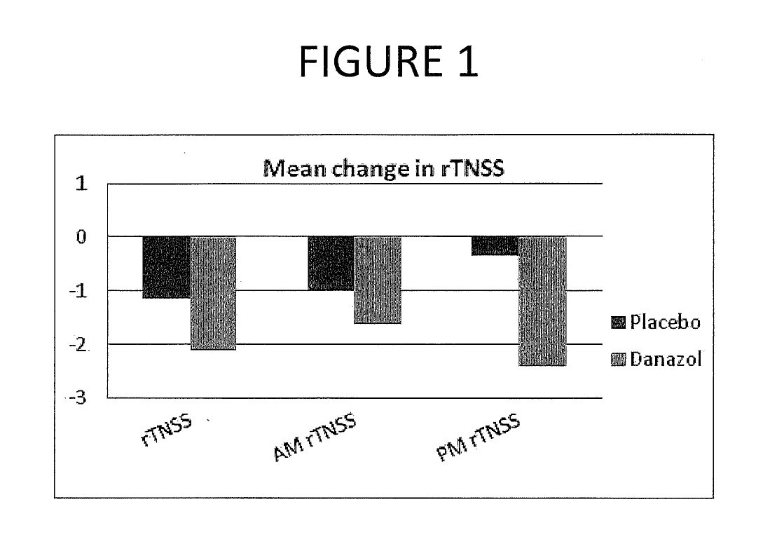

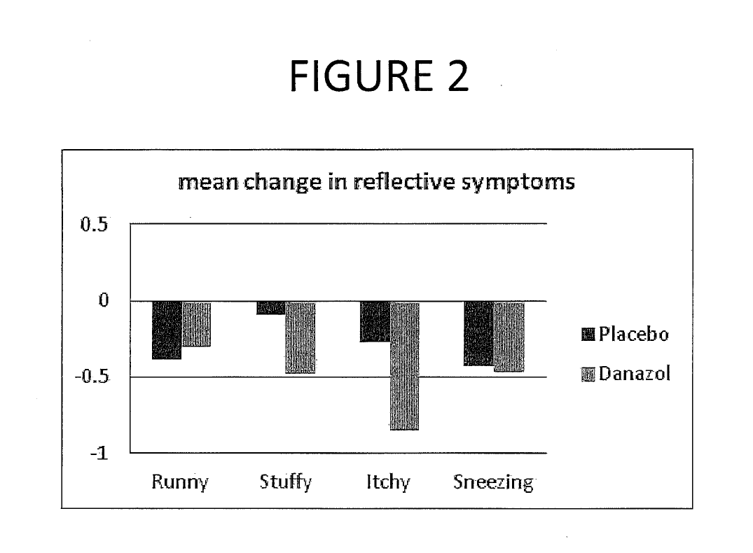

The change in efficacy was calculated as the change in the double-blind value—baseline value as follows:
Change in rTNSS: mean double-blind rTNSS—mean baseline rTNSS
Change in iTNSS: mean double-blind iTNSS—mean baseline iTNSS Student's t-test: mean (SD) difference between treatment groups for the following:
Mean change in (rTNSS/iTNSS)
Mean change in AM (rTNSS/iTNSS)
Mean change in PM (rTNSS/iTNSS)
Mean change in individual symptom scores for each of the four nasal symptoms.
   The results obtained by analysis using the Student's t-test are shown in FIGS. 1-4 and Tables 1-5 and are summarized below (number of subjects screened: n=21; number of subjects randomized: n=20; placebo, n=10 (ITT population) and Danazol, n=10 (ITT population):
rTNSS: Danazol showed a 2.11 point decrease in reflective symptom severity compared to 1.16 point decrease with placebo (Table 2: rTNSS and FIG. 1). The effect was even greater during the PM assessment: −2.39 with danazol versus −0.34 with placebo (Table 2: AM rTNSS and PM rTNSS and FIG. 1). The largest improvements were with itchy and stuffy nose symptoms (Table 3 and FIG. 2). 6/7 efficacy measures showed a larger improvement with danazol than placebo (all assessments but runny nose symptom).

TABLE 1
Baseline efficacy variables, mean (SD)

| Efficacy | | | T-test |
|---|---|---|---|
| Variable | Placebo | Danazol | p value |
| rTNSS | 6.50 (1.30) | 7.55 (2.63) | 0.28 |
| iTNSS | 6.25 (1.56) | 6.76 (2.52) | 0.59 |

TABLE 2
Mean change in rTNSS

| Efficacy | Placebo | Danazol | T-test |
|---|---|---|---|
| Variable | Mean (SD) | Mean (SD) | p value |
| rTNSS | −1.16 (2.56) | −2.11 (2.66) | 0.43 |
| AM rTNSS | −0.98 (2.38) | −1.62 (2.40) | 0.56 |
| PM rTNSS | −0.34 (2.68) | −2.39 (2.68) | 0.10 |

TABLE 3
Mean Change in Reflective Symptoms

| | Placebo | Danazol | T-test |
|---|---|---|---|
| Symptom | Mean (SD) | Mean (SD) | p value |
| rRunny nose | −0.38 (0.69) | −0.30 (0.66) | 0.80 |
| rStuffy nose | −0.09 (1.03) | −0.48 (0.69) | 0.33 |
| rItchy nose | −0.27 (0.74) | −0.85 (1.02) | 0.17 |
| rSneezing | −0.43 (0.61) | −0.47 (0.71) | 0.88 |

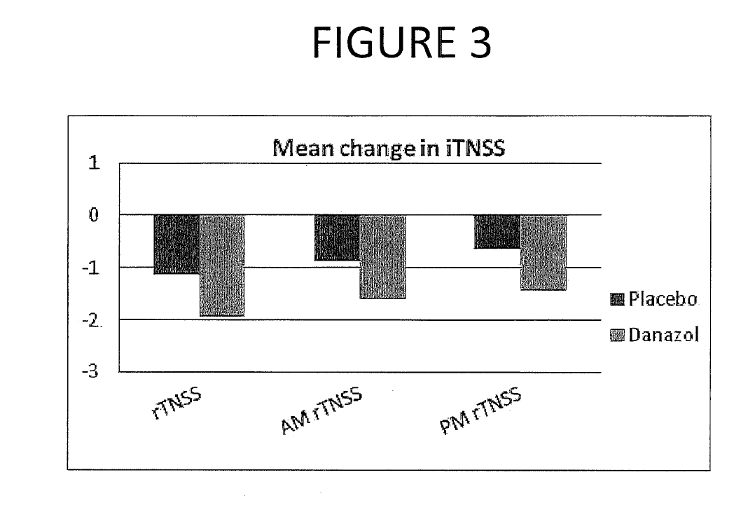

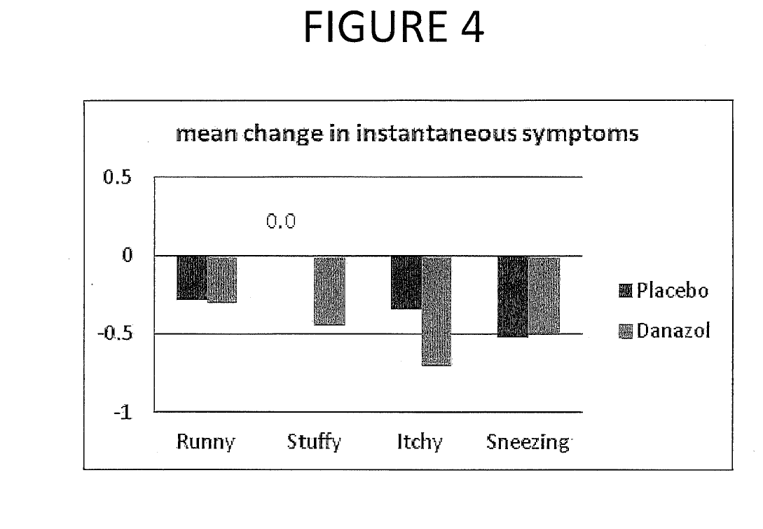

iTNSS: Similar improvements were seen with instantaneous symptom improvements as with reflective symptom improvements. Danazol showed a 1.94 point decrease in instantaneous symptom severity compared to 1.14 point decrease with placebo (Table 4: iTNSS and FIG. 3). The similar effects were seen during the AM and PM assessment (Table 4: AM iTNSS and PM iTNSS and FIG. 3). The largest improvements were with itchy and stuffy nose symptoms (Table 5 and FIG. 4). 6/7 efficacy measures showed a larger improvement with danazol than with placebo (all assessments but sneezing).

TABLE 4
Mean Change in iTNSS

| Efficacy | Placebo | Danazol | T-test |
|---|---|---|---|
| variable | Mean (SD) | Mean (SD) | p value |
| iTNSS | −1.14 (2.50) | −1.94 (2.68) | 0.50 |
| AM iTNSS | −0.88 (2.45) | −1.59 (2.36) | 0.52 |
| PM iTNSS | −0.63 (2.46) | −1.42 (2.64) | 0.50 |

TABLE 5
Mean Change in Instantaneous Symptoms

| | Placebo | Danazol | T-test |
|---|---|---|---|
| Symptom | Mean (SD) | Mean (SD) | p value |
| iRunny nose | −0.28 (0.71) | −0.30 (1.07) | 0.97 |
| iStuffy nose | 0.00 (1.05) | −0.44 (0.56) | 0.27 |
| iItchy nose | −0.34 (0.71) | −0.70 (0.92) | 0.35 |
| iSneezing | −0.52 (0.38) | −0.50 (0.53) | 0.95 |

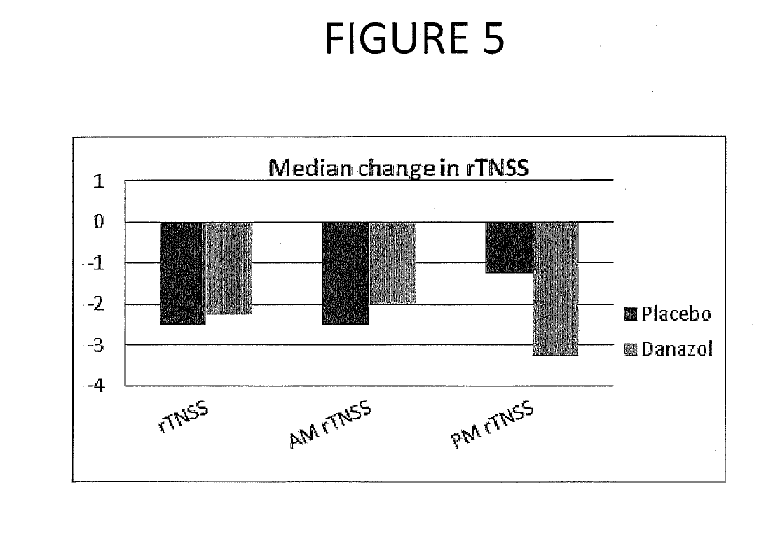

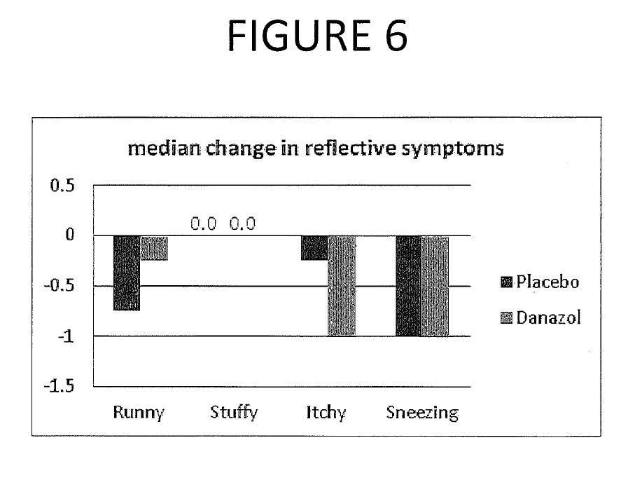

The data were also analyzed using the Wilcoxon rank-sum test: median (IQR) difference between treatment groups for the following (number of subjects screened: n=21; number of subjects randomized: n=20; placebo, n=10 (ITT population) and Danazol, n=10 (ITT population):
Median change in (rTNSS/iTNSS)
Median change in AM (rTNSS/iTNSS)
Median change in PM (rTNSS/iTNSS)
Median change in individual symptom scores for reach of the four nasal symptoms.
   The results obtained by analysis using the Wilcoxon rank-sum test are presented in FIGS. 5-9 as well as in Tables 6-10 below.
rTNSS: Danazol showed a 3.25 point decrease in reflective symptom severity compared to 1.25 point decrease with placebo during the PM assessment (Table 7: rTNSS and FIG. 5). The largest improvements were with itchy nose symptoms (Table 8 and FIG. 6).

TABLE 6
Baseline efficacy variables, median (IQR)

| Efficacy | | | | Wilcoxon |
|---|---|---|---|---|
| Variable | Placebo | | Danazol | p value |
| rTNSS | 6.0 | (5-7) | 5.75 (5-9) | 0.91 |
| iTNSS | 6.0 | (5-6.5) | 5.75 (5-8) | 0.63 |

TABLE 7
Mean change in rTNSS

| Efficacy | Placebo | | Danazol | | Wilcoxon |
|---|---|---|---|---|---|
| Variable | Median (IQR) | | Median (IQR) | | p value |
| rTNSS | −2.5 | (−3.0-2.0) | −2.25 | (−4.5-0.5) | 0.76 |
| AM rTNSS | −2.5 | (−3.0-1.5) | −2.0 | (−4.0-1.0) | 0.82 |
| PM rTNSS | −1.25 | (−3.0-3.00) | −3.25 | (−4.0-0.0) | 0.17 |

TABLE 8
Mean Change in Reflective Symptoms

| | Placebo | | Danazol | | Wilcoxon |
|---|---|---|---|---|---|
| Symptom | Median (IQR) | | Median (IQR) | | p value |
| rRunny nose | −0.75 | (−1.0-0.0) | −0.25 | (−1.0-0.0) | 0.51 |
| rStuffy nose | 0.0 | (−1.0-1.0) | 0.0 | (−1.0-0.0) | 0.44 |
| rItchy nose | −0.25 | (−1.0-0.0) | −1.0 | (−1.0-0.0) | 0.23 |
| rSneezing | −1.0 | (−1.0-0.0) | −1.0 | (−1.0-0.0) | 0.74 |

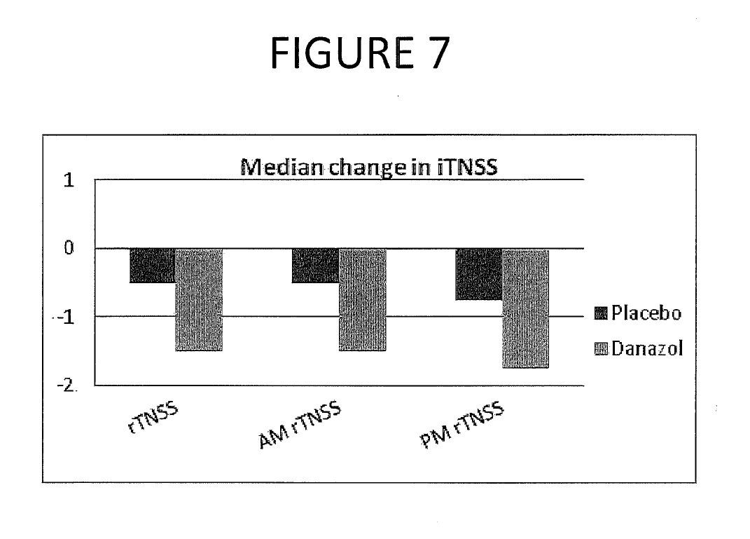

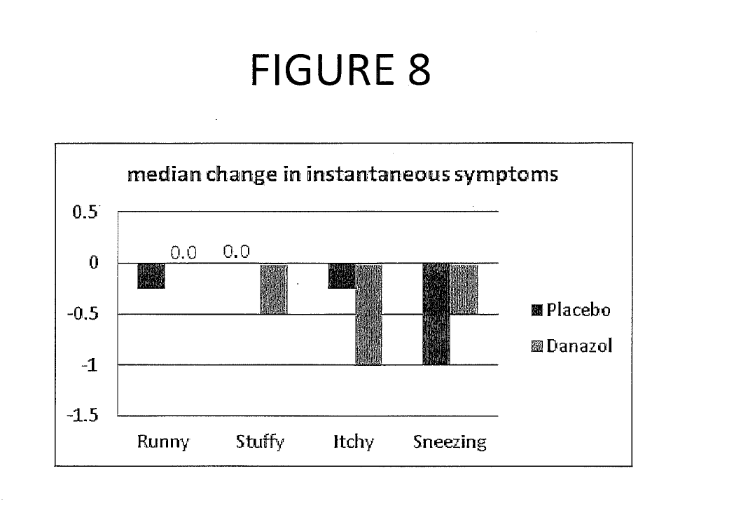

iTNSS: Danazol showed a 1.75 point decrease in instantaneous symptom severity compared to 0.75 point decrease with placebo (Table 9: iTNSS and FIG. 7). Similar effects were seen during the AM and PM assessment (Table 9: AM iTNSS and PM iTNSS and FIG. 7). The largest improvements were with itchy and stuffy nose symptoms (Table 10 and FIG. 8).

TABLE 9
Mean Change in iTNSS

| Efficacy | Placebo | | Danazol | | Wilcoxon |
|---|---|---|---|---|---|
| variable | Median (IQR) | | Median (IQR) | | p value |
| iTNSS | −0.75 | (−2.5-0.0) | −1.75 | (−2.5--−0.5) | 0.44 |
| AM iTNSS | −0.5 | (−3.0-0.5) | −1.5 | (−2.0-0.0) | 0.52 |
| PM iTNSS | −0.5 | (−3.0-2.0) | −1.5 | (−2.5-0.0) | 0.63 |

TABLE 10
Mean Change in Instantaneous Symptoms

| | Placebo | | Danazol | | Wilcoxon |
|---|---|---|---|---|---|
| Symptom | Median (IQR) | | Median (IQR) | | p value |
| iRunny nose | −0.25 | (−1.0-0.0) | 0.0 | (−1.0-0.0) | 0.56 |
| iStuffy nose | 0.0 | (−0.5-1.0) | −0.5 | (−1.0-0.0) | 0.06 |
| iItchy nose | −0.25 | (−1.0-0.0) | −1.0 | (−1.0--−0.5) | 0.17 |
| iSneezing | −1.0 | (−1.0-0.0) | −0.5 | (1.0-0.0) | 0.42 |

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

What is claimed:

1. A method of treating rhinitis comprising administering an effective amount of a danazol compound or a pharmaceutically acceptable salt thereof, to an animal in need thereof.

2. The method of claim 1, wherein the rhinitis is allergic rhinitis.

3. The method of claim 1, wherein the effective amount of the danazol compound or the pharmaceutically acceptable salt thereof is from about 100 μg to about 3000 μg per day.

4. The method of claim 1, wherein the effective amount of the danazol compound or the pharmaceutically acceptable salt thereof is from about 500 μg to about 1500 μg per day.

5. The method of claim 1, wherein administration of the danazol compound or a pharmaceutically acceptable salt thereof, is commenced within 24 hours of diagnosis of rhinitis.

6. The method of claim 1, wherein administration of the danazol compound or a pharmaceutically acceptable salt thereof, is commenced at the appearance of one or more early signs of, or a predisposition to develop, rhinitis.

7. The method of claim 6, wherein the one or more early signs of rhinitis are selected from the group consisting of rhinorrhea, nasal congestion, nasal itching and sneezing.

8. The method of claim 1, wherein the danazol compound is danazol.

9. The method of claim 1, wherein the danazol compound or a pharmaceutically acceptable salt thereof is administered in combination with a second drug suitable for treating rhinitis.

10. The method of claim 9, wherein the second drug suitable for treating rhinitis is selected from the group consisting of antihistamines, decongestants, anti-inflammatories, mast cell stabilizers, leukotriene modifiers and IgE blockers.

11. A pharmaceutical product, comprising a danazol compound or a pharmaceutically-acceptable salt thereof formulated for administration by a route selected from the group consisting of inhalation, insufflation and nasal administration to the nose.

12. The pharmaceutical product of claim 11, wherein the danazol compound or a pharmaceutically-acceptable salt thereof formulated for administration by inhalation is packaged in a device selected from the group consisting of insufflators, nebulizers, pressurized packs, squeeze bottle, a syringe, a dropper, a spray device, an atomizer device, and an aerosolizer.

13. The pharmaceutical product of claim 12, wherein the pressurized pack comprises a propellant selected from the group consisting of dichlorodifluoromethane, trichlorofluoromethane, dichlorotetrafluoroethane, and carbon dioxide.

14. The pharmaceutical product of claim 11, wherein the danazol compound or a pharmaceutically-acceptable salt thereof formulated for administration by inhalation or insufflation comprises a powder mix of the danazol compound or pharmaceutically-acceptable salt thereof and a powder base.

15. The pharmaceutical product of claim 14, wherein the powder mix is in a dosage form selected from the group consisting of capsules, cartridges, gelatin packs and blister packs.

16. The pharmaceutical product of claim 15, wherein the powder mix is delivered by a device selected from the group consisting of an inhalator, insufflator and metered-dose inhaler.

17. The pharmaceutical product of claim 11, wherein the danazol compound or a pharmaceutically-acceptable salt thereof formulated for nasal administration is in a form of drops or sprays.

18. The pharmaceutical product of claim 17, wherein the danazol compound or a pharmaceutically-acceptable salt thereof comprises about 0.1% (w/v).

19. The pharmaceutical product of claim 17, wherein the drops or sprays are contained within an intranasal delivery system.

20. The pharmaceutical product of claim 17, wherein the intranasal delivery system comprises an atomizing device.

21. The pharmaceutical product of claim 20, wherein the atomizing device comprises a bottle and a pump.

22. The pharmaceutical product of claim 21, wherein the pump is a metered dose pump.

23. The pharmaceutical product of claim 22, wherein the metered dose pump delivers an intranasal volume of the danazol compound or a pharmaceutically-acceptable salt thereof of about 0.15 ml per pump.

24. The pharmaceutical product of claim 17, further comprising an aqueous or non-aqueous base comprising one or more agents selected from the group consisting of dispersing agents, solubilizing agents, and suspending agents.

25. The pharmaceutical product of claim 11, wherein the danazol compound or a pharmaceutically-acceptable salt thereof formulated for nasal administration is in a form selected from the group consisting of ointments, gels and creams.

26. The pharmaceutical product of claim 25, wherein the danazol compound or a pharmaceutically-acceptable salt thereof further comprises excipients selected from the group consisting of animal fats, vegetable fats, oils, waxes, paraffins, starch, tragacanth, cellulose derivatives, polyethylene glycol, silicones, bentonites, silicic acid, talc, zinc oxide and mixtures thereof.

27. The pharmaceutical product of claim 11, further comprising an absorption or permeation enhancer.

28. The pharmaceutical product of claim 11, further comprising a thickening agent or viscosity enhancer to increase the residence time of the danazol compound or pharmaceutically-acceptable salt thereof in the nose.

29. The pharmaceutical product of claim 11, further comprising a pharmaceutically-acceptable carrier.

30. The pharmaceutical product of claim 11, wherein the danazol compound is danazol.
